SEQUENCE LISTING (58 sequences)

SEQ ID NO: 1 - protein, 14 aa, Artificial Sequence
  synthetic Arabidopsis Ethylene Response Factor (ERF) Group VII transcription factor RELATED TO AP2 (RAP) RAP2.12 N-terminal MC-dipeptide
MCGGAIISDFIPPP

SEQ ID NO: 2 - protein, 14 aa, Artificial Sequence
  synthetic Arabidopsis Ethylene Response Factor (ERF) Group VII transcription factor HYPOXIA RESPONSIVE (HRE) HRE1 N-terminal MC-dipeptide
MCGGAVISDYIAPE

SEQ ID NO: 3 - protein, 14 aa, Artificial Sequence
  synthetic Arabidopsis Ethylene Response Factor (ERF) Group VII transcription factor HYPOXIA RESPONSIVE (HRE) HRE2 N-terminal MC-dipeptide
MCGGAIISDFIWSK

SEQ ID NO: 4 - protein, 14 aa, Artificial Sequence
  synthetic Arabidopsis Ethylene Response Factor (ERF) Group VII transcription factor RELATED TO AP2 (RAP) RAP2.2 N-terminal MC-dipeptide
MCGGAIISDFIPPP

SEQ ID NO: 5 - protein, 12 aa, Artificial Sequence
  synthetic Arabidopsis Ethylene Response Factor (ERF) Group VII transcription factor RELATED TO AP2 (RAP) RAP2.3 N-terminal MC-dipeptide
MCGGAIISDYAP

SEQ ID NO: 6 - protein, 14 aa, Artificial Sequence
  synthetic rice Ethylene Response Factor (ERF) Group X transcription factor Os11g06770.2 N-terminal MC-dipeptide
MCGGGKVASPPGPR

SEQ ID NO: 7 - protein, 14 aa, Artificial Sequence
  synthetic rice Ethylene Response Factor (ERF) transcription factor Os06g43220.1 N-terminal MC-dipeptide
MCRDCGKQVYLGGF

SEQ ID NO: 8 - protein, 14 aa, Artificial Sequence
  synthetic rice Ethylene Response Factor (ERF) Group III transcription factor Os08g43210.1 N-terminal MC-dipeptide
MCTSKLEEITGEWP

SEQ ID NO: 9 - protein, 13 aa, Artificial Sequence
  synthetic rice Ethylene Response Factor (ERF) Group III transcription factor Os09g35030.1 N-terminal MC-dipeptide
MCGIKQEMSGESS

SEQ ID NO: 10 - protein, 14 aa, Artificial Sequence
  synthetic rice Ethylene Response Factor (ERF) Group VII transcription factor Os03g08490.1 N-terminal MC-dipeptide
MCGGAILAEFIPAP

SEQ ID NO: 11 - protein, 14 aa, Artificial Sequence
  synthetic rice Ethylene Response Factor (ERF) Group VII transcription factor Os03g08500.1 N-terminal MC-dipeptide
MCGGAILAELIPSA

SEQ ID NO: 12 - protein, 14 aa, Artificial Sequence
  synthetic rice Ethylene Response Factor (ERF) Group VII transcription factor Os03g08470.1 N-terminal MC-dipeptide
MCGGAILANIIPAT

SEQ ID NO: 13 - protein, 14 aa, Artificial Sequence
  synthetic rice Ethylene Response Factor (ERF) Group VII transcription factor Os09g26420.1 N-terminal MC-dipeptide
MCGGAIISGFIPPS

SEQ ID NO: 14 - protein, 14 aa, Artificial Sequence
  synthetic rice Ethylene Response Factor (ERF) Group VII transcription factor Os06g09390.1 N-terminal MC-dipeptide
MCGGAILSDLIPPP

SEQ ID NO: 15 - protein, 14 aa, Artificial Sequence
  synthetic rice Ethylene Response Factor (ERF) Group VII transcription factor Os02g54160.1 N-terminal MC-dipeptide
MCGGAIIHHLKGHP

SEQ ID NO: 16 - protein, 12 aa, Artificial Sequence
  synthetic rice Ethylene Response Factor (ERF) Group VII transcription factor Os03g22170.1 N-terminal MC-dipeptide
MCGGAIPLISSR

SEQ ID NO: 17 - protein, 14 aa, Artificial Sequence
  synthetic rice Ethylene Response Factor (ERF) Group VII transcription factor Os07g47790.1 N-terminal MC-dipeptide
MCGGAIISDFIPQR

SEQ ID NO: 18 - protein, 14 aa, Artificial Sequence
  synthetic rice Ethylene Response Factor (ERF) Group VII transcription factor Os01g21120.1 N-terminal MC-dipeptide
MCGGAIIYDYIPAR

SEQ ID NO: 19 - protein, 14 aa, Artificial Sequence
  synthetic rice Ethylene Response Factor (ERF) Group VII transcription factor Os05g29810.1 N-terminal MC-dipeptide
MCGGAIIADFVPPA

SEQ ID NO: 20 - protein, 14 aa, Artificial Sequence
  synthetic rice Ethylene Response Factor (ERF) Group XI transcription factor Os12g41060.1 N-terminal MC-dipeptide
MCGGGPDNHHAITV

SEQ ID NO: 21 - protein, 14 aa, Artificial Sequence
  synthetic Arabidopsis Ethylene Response Factor (ERF) Group VII transcription factor SNORKEL1 N-terminal MC-dipeptide
MCGGCLIPDELVGK

SEQ ID NO: 22 - protein, 14 aa, Artificial Sequence
  synthetic Arabidopsis Ethylene Response Factor (ERF) Group VII transcription factor SNORKEL2 N-terminal MC-dipeptide
MCGENDNNGAAAGS

SEQ ID NO: 23 - protein, 14 aa, Artificial Sequence
  synthetic Arabidopsis Ethylene Response Factor (ERF) Group VII transcription factor SUBMERGENCE (SUB) SUB1A-1 N-terminal MC-dipeptide
MCGGEVIPADMPAA

SEQ ID NO: 24 - protein, 14 aa, Artificial Sequence
  synthetic Arabidopsis Ethylene Response Factor (ERF) Group VII transcription factor SUBMERGENCE (SUB) SUB1B-1 N-terminal MC-dipeptide
MCGGALIPNDYGDK

SEQ ID NO: 25 - protein, 5 aa, Artificial Sequence
  synthetic Arabidopsis Ethylene Response Factor (ERF) Group VII transcription factor SUBMERGENCE (SUB) SUB1C-1 N-terminal MC-dipeptide
MRRRV

SEQ ID NO: 26 - protein, 7 aa, Artificial Sequence
  synthetic Arabidopsis Ethylene Response Factor (ERF) transcription factor N-terminal MC-dipeptide
MCGGAII

SEQ ID NO: 27 - protein, 14 aa, Artificial Sequence
  synthetic MADS AFFECTING FLOWERING 5 (MAF5) N-terminal sequence
MCRKSEAMGRRRVE

SEQ ID NO: 28 - protein, 14 aa, Artificial Sequence
  synthetic REDUCED VERNALIZATION RESPONSE 2 (VRN2) N-terminal sequence
MCRQNCRAKSSPEE

SEQ ID NO: 29 - DNA, 22 bp, Artificial Sequence
  synthetic Actin2_F primer
atggctgaggctgatgatattc

SEQ ID NO: 30 - DNA, 24 bp, Artificial Sequence
  synthetic Actin2_R primer
agaaacattttctgtgaacgattc

SEQ ID NO: 31 - DNA, 20 bp, Artificial Sequence
  synthetic 3xHA_R primer
agagtactgctagcggctta

SEQ ID NO: 32 - DNA, 30 bp, Artificial Sequence
  synthetic MAF5_MC_F primer
aaaaacgcgtatgtgtcggaagagtgaagc

SEQ ID NO: 33 - DNA, 30 bp, Artificial Sequence
  synthetic MAF5_MA_F primer
aaaaacgcgtatggctcggaagagtgaagc

SEQ ID NO: 34 - DNA, 20 bp, Artificial Sequence
  synthetic MAF5_R primer
ttacttgagaagcgggagag

SEQ ID NO: 35 - DNA, 31 bp, Artificial Sequence
  synthetic VRN2_MC_F primer
aaaaacgcgtatgtgtaggcagaattgtcgc

SEQ ID NO: 36 - DNA, 31 bp, Artificial Sequence
  synthetic VRN2_MA_F primer
aaaaacgcgtatggctaggcagaattgtcgc

SEQ ID NO: 37 - DNA, 22 bp, Artificial Sequence
  synthetic VRN2_R primer
ttacttgtctctgctgttattg

SEQ ID NO: 38 - DNA, 29 bp, Artificial Sequence
  synthetic HRE1_MC_F primer
aaaggatccatgtgcggaggagctgtaat

SEQ ID NO: 39 - DNA, 29 bp, Artificial Sequence
  synthetic HRE1_MA_F primer
aaaggatccatggccggaggagctgtaat

SEQ ID NO: 40 - DNA, 29 bp, Artificial Sequence
  synthetic HRE1_R primer
aaatctagatcaggaccatagacccatgt

SEQ ID NO: 41 - DNA, 29 bp, Artificial Sequence
  synthetic HRE2_MC_F primer
aaaggatccatgtgtgggggagctatcat

SEQ ID NO: 42 - DNA, 29 bp, Artificial Sequence
  synthetic HRE2_MA_F primer
aaaggatccatggctgggggagctatcat

SEQ ID NO: 43 - DNA, 31 bp, Artificial Sequence
  synthetic HRE2_R primer
aaatctagattaattggagtcttgatagctc

SEQ ID NO: 44 - DNA, 32 bp, Artificial Sequence
  synthetic RAP2.12_MC_F primer
aaaggatccatgtgtggaggagctataatatc

SEQ ID NO: 45 - DNA, 32 bp, Artificial Sequence
  synthetic RAP2.12_MA_F primer
aaaggatccatggctggaggagctataatatc

SEQ ID NO: 46 - DNA, 31 bp, Artificial Sequence
  synthetic RAP2.12_R primer
aaatctagatcagaagactcctccaatcatg

SEQ ID NO: 47 - DNA, 30 bp, Artificial Sequence
  synthetic RAP2.2_MC_F primer
aaaggatccatgtgtggaggagctataatc

SEQ ID NO: 48 - DNA, 30 bp, Artificial Sequence
  synthetic RAP2.2_MA_F primer
aaaggatccatggctggaggagctataatc

SEQ ID NO: 49 - DNA, 31 bp, Artificial Sequence
  synthetic RAP2.2_R primer
aaaatctagatcaaaagtctccttccagcat

SEQ ID NO: 50 - DNA, 30 bp, Artificial Sequence
  synthetic EBP_MC_F primer
aaaggatccatgtgtggcggtgctattatt

SEQ ID NO: 51 - DNA, 30 bp, Artificial Sequence
  synthetic EBP_MA_F primer
aaaggatccatggctggcggtgctattatt

SEQ ID NO: 52 - DNA, 30 bp, Artificial Sequence
  synthetic EBP_R primer
aaatctagattactcatacgacgcaatgac

SEQ ID NO: 53 - DNA, 31 bp, Artificial Sequence
  synthetic SUB1A_MC_F primer
aaaagaattcatgtgtggaggagaagtgatc

SEQ ID NO: 54 - DNA, 31 bp, Artificial Sequence
  synthetic SUB1A_MA_F primer
aaaagaattcatggctggaggagaagtgatc

SEQ ID NO: 55 - DNA, 31 bp, Artificial Sequence
  synthetic SUB1A_E5A_F primer
aaaagaattcatgtgtggaggagctgtgatc

SEQ ID NO: 56 - DNA, 25 bp, Artificial Sequence
  synthetic SUB1A_R primer
aaaaggtaccccctgcatatgatat

SEQ ID NO: 57 - DNA, 28 bp, Artificial Sequence
  synthetic SUB1C_F primer
aaaagaattcatgcgccgccgcgtctcc

SEQ ID NO: 58 - DNA, 29 bp, Artificial Sequence
  synthetic SUB1C_R primer
aaaaggtaccgctccagaagcgcatgtcg

(19) United States
(12) Patent Application Publication
(10) Pub. No.: US 2017/0088851 A1
(43) Pub. Date: Mar. 30, 2017

(54) METHODS FOR IMPROVING ABIOTIC STRESS RESPONSE

(71) Applicants: The Regents of the University of California, Oakland, CA (US); The University of Nottingham, Nottingham (GB); The University of Sheffield, Sheffield (GB)

(72) Inventors: Julia Baily-Serres, Riverside, CA (US); Michael Holdsworth, Loughborough (GB); Julie Gray, Sheffield (GB)

(21) Appl. No.: 15/230,130

(22) Filed: Aug. 5, 2016

Related U.S. Application Data
(62) Division of application No. 13/656,172, filed on Oct. 19, 2012, now abandoned.
(60) Provisional application No. 61/550,329, filed on Oct. 21, 2011.

(51) Int. Cl. C12N 15/82; C07K 14/415

(52) U.S. Cl. CPC C12N 15/8271; C12N 15/8251; C12N 15/8273; C12N 15/8267; C07K 14/415

(57) ABSTRACT

Provided are methods and compositions for improving the growth characteristics of plants.

### CROSS REFERENCES TO RELATED APPLICATIONS

The present application is a divisional of U.S. patent application Ser. No. 13/656,172, filed Oct. 19, 2012, which claims priority to U.S. Provisional Application No. 61/550,329, filed Oct. 21, 2011, the disclosure of which is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under Grant No. NSF-IOS-0750811, awarded by the National Science Foundation and Grant No. NIFA 2008-35100-04528 awarded by the National Institute of Food and Agriculture. The Government has certain rights in the invention.

### BACKGROUND OF THE INVENTION

Plants and animals are obligate aerobes, requiring oxygen for mitochondrial respiration and energy production. In plants, an unanticipated decline in oxygen availability (hypoxia), as caused by root waterlogging or foliage submergence, triggers changes in gene transcription and mRNA translation that promote anaerobic metabolism and thus sustain substrate-level ATP production¹. In contrast to animals², direct oxygen sensing has not been ascribed to a mechanism of gene regulation in response to oxygen deprivation in plants. Here we show that the N-end rule pathway of targeted proteolysis (NERP) acts as a homeostatic sensor of severe low oxygen in Arabidopsis, through its regulation of key hypoxia response transcription factors. We found that plants lacking components of the NERP constitutively express core hypoxia response genes and are more tolerant of hypoxic stress. We identify the hypoxia-associated Ethylene Response Factor (ERF) Group VII transcription factors of Arabidopsis as substrates of this pathway. Regulation of these proteins by the NERP occurs through a characteristic conserved motif at the N-terminus initiating with MC-. Enhanced stability of one of these proteins, HRE2, under low oxygen conditions improves hypoxia survival and reveals a molecular mechanism for direct oxygen sensing in plants via the evolutionarily conserved NERP. SUB1A-1, a major determinant of submergence tolerance in rice³, was shown not to be a substrate for the NERP despite containing the N-terminal motif, suggesting that it is uncoupled from NERP regulation, and that enhanced stability may relate to the superior tolerance of Sub1 rice varieties to multiple abiotic stresses⁴.

### SUMMARY OF THE INVENTION

In a first aspect, the present invention provides a method of improving plant growth characteristics comprising the steps of:

(i) disrupting the N-end rule pathway of targeted proteolysis in the plant; and

(ii) optionally selecting for plants having improved growth characteristics.

The improved plant growth characteristics may be one or more selected from the group comprising: resistance to oxygen stress/hypoxia; tolerance to drought conditions; improved water use efficiency; lower stomal density and/or index; improved germination performance; improved dormancy performance; increased or decreased ABA sensitivity; and improved resistance to abiotic stresses in general. Preferably improved is determined relative to the characteristics in a control plant.

Improved resistance to oxygen stress may result in a 10%, 20%, 30%, 40%, 50% or more increase in survival score when a plant is subjected to 9 hours or more of oxygen deprivation, compared to a control plant.

Improved tolerance to drought conditions or improved water use efficiency may result in 10%, 20%, 30%, 40%, 50%, 60%, 70% 80% or more increase in relative water content under drought conditions compared to a control plant.

Lower stomal index may be a reduction in stomal index of 10%, 20%, 30%, 40% or more compared to a control plant.

The N-end rule pathway of targeted proteolysis may be disrupted in many different ways. The N-end rule pathway of targeted proteolysis may be disrupted in one or more of the following ways: by reducing or eliminating the ability of the N-end rule pathway of targeted proteolysis to degrade proteins initiating with the MC-motif at the N-terminus; modifying MC-motif proteins so that they are no longer substrates for the N-end rule pathway of targeted proteolysis; by increasing the level of expression of the substrates of the N-end rule pathway of targeted proteolysis such that the level of un-degraded protein is elevated; and by altering the level of oxygen and/or nitric oxide to which the plant is exposed. Preferably by controlling the level of oxygen or nitric oxide the rate of degradation of MC-motif proteins in the plant can be controlled.

The ability of the N-end rule pathway of targeted proteolysis to degrade proteins initiating with the MC-motif at the N-terminus may be reduced or eliminated by reducing or eliminating the expression of functional proteins involved in the process of degrading proteins carrying an MC-motif at the N-terminal end. This may be achieved by introducing a mutation into one or more to the genes selected from the group comprising PRT1, PRT6, ATE1 and ATE2 or a homolog or species ortholog thereof.

MC-motif proteins may be modified so that they are not substrates for the N-end rule pathway of targeted proteolysis by altering the MC-motif, for example by mutating the cysteine residue to an alternative amino acid, for example alanine. Such modified proteins may be referred to as stabilized.

The MC-motif protein may be an ethylene response factor (ERF) Group VII transcription factor, such as, one or more of HRE1, HRE2, RAP2.12, RAP2.2 and RAP2.3(EBP), or a homolog or species ortholog thereof.

In a preferred embodiment the method of improving plant growth characteristics comprises the steps of:

(i) stabilising one or more of the ethylene response factor (ERF) Group VII transcription factors, such as, one or more of HRE1, HRE2, RAP2.12, RAP2.2 and RAP2.3(EBP), or a homolog or a species ortholog thereof; and

(ii) optionally selecting for plants having improved growth characteristics.

Preferably in this embodiment the improved plant growth characteristic is selected from increased resistance to oxygen stress/hypoxia; increased tolerance to drought conditions; improved water use efficiency; and lower stromal density and/or index.

Preferably the improved stability of the one or more of the ethylene response factor (ERF) Group VII transcription factors, is achieved by mutating the MC-N-terminal motif such that is not a signal for degradation by the N-end rule pathway of targeted proteolysis. Preferably the C amino acid of the MC-motif is altered. The C amino acid may be mutated to alanine. The stabilised ERF protein may be HRE1^{C2A}, HRE2^{C2A}, RAP2.12^{C2A}, RAP2.2^{C2A}, and RAP2.3^{C2A} a homolog or species ortholog thereof. Preferably if the desired improved plant characteristic is improved tolerance to oxygen deprivation then either or both or HRE1 and HRE2, or a homolog or species ortholog thereof, are mutated. If the desired improved plant characteristic is improved germination, preferably by reduced sensitivity to ABA, then one or more of RAP2.12, RAP2.2 and RAP2.3, or a homolog or species ortholog thereof, may be deleted or mutated to no longer be a substrate for the N-end rule pathway of targeted proteolysis.

The N-end rule pathway of targeted proteolysis may be disrupted by mutating endogenous DNA, for example by mutating the PRT1 or PRT6 gene, a homolog or species ortholog thereof, so it is not functional or has reduced function; or by mutating endogenous DNA encoding MC-motif proteins so that one or more mutated MC-motif proteins, such as one or more ERF proteins, are produced with a mutated N-terminal MC-motif and so cannot to be recognized by the N-end rule pathway of targeted proteolysis. Alternatively the N-end rule pathway of targeted proteolysis may be disrupted by introducing exogenous DNA, such as a gene encoding one or more ERF proteins, or a homolog or species ortholog thereof, which encode proteins which have altered MC-N-terminal motifs such that the expressed proteins are not substrates for the N-end rule pathway of targeted proteolysis. In this way the MC-motif protein, such as an ERF protein, concentration will increase in the plant.

The invention further provides plants obtained or obtainable by the method of the invention.

According to a further aspect the invention provides a genetically modified plant or plant cell comprising improved plant growth characteristics comprising a disrupted N-end rule pathway of targeted proteolysis.

The improved plant growth characteristics may be as described above, and the N-end rule pathway of targeted proteolysis may be disrupted as described above.

In an embodiment the endogenous DNA of the plant or plant cell may be modified to effect disruption of the N-end rule pathway of targeted proteolysis. For example, one or more of the PRT1, PRT6, ATE1 and/or ATE2 genes, a homolog or species ortholog thereof, may be modified to reduce or eliminate the activity of the N-end rule pathway of targeted proteolysis.

Alternatively, or additionally, one or more of the endogenous ERF genes, or a homolog or species ortholog thereof, may be mutated to prevent them from being substrates for the N-end rule pathway of targeted proteolysis. Preferably this will allow accumulation, or an increase in concentration, of the expression product of these genes. Such mutations are described in detail above.

According to another aspect the invention provides an expression cassette comprising a promoter operably linked to a polynucleotide encoding a stabilised form of one or more of the ethylene response factor (ERF) Group VII transcription factors, such as, one or more of HRE1, HRE2, RAP2.12, RAP2.2 and RAP2.3(EBP), or a homolog or species ortholog thereof. The promoter may be a constitutive promoter, or may be a promoter intended to be expressed at a particular point in development of a plant, a plant cell or a seed. The promoter may be tissue specific. The polynucleotide may be heterologous to the intended host plant.

According to yet another aspect the invention provides an expression vector comprising the expression cassette according to the invention.

According to a further aspect the invention provides a plant, a plant cell or a seed transformed with an expression cassette or expression vector according to the invention. Preferably the expression cassette is stably integrated into the host.

According to another aspect the invention provides a method of producing a transgenic plant cell, plant or part thereof with improved plant growth characteristics comprising the steps of:

(i) introducing into a plant cell an expression cassette or expression vector according to the invention;

(ii) generating from the plant cell a transgenic plant expressing the polynucleotide encoded by the expression cassette.

According to a still further aspect the invention provides a transgenic plant, plant cell or seed with improved plant growth characteristics comprising elevated expression levels of one or more ERF proteins, or homolog or species ortholog thereof; or expression of a stabilised form of one or more ERF proteins, or homolog or species ortholog thereof, which have reduced or no susceptibility to degradation by the N-end rule pathway of targeted proteolysis

According to a yet further aspect the invention provides a transgenic plant, plant cell or seed with elevated expression of one or more ERF proteins, or homolog or species ortholog thereof; or expression of a stabilised form of one or more ERF proteins, or homolog or species ortholog thereof, which have reduced or no susceptibility to degradation by the N-end rule pathway of targeted proteolysis.

According to another aspect the invention provides a seed or plant in which one or more of the ERF proteins, or homolog or species ortholog thereof, are stabilised such that they have reduced or no susceptibility to degradation by the N-end rule pathway of targeted proteolysis and the seed or plant displays increased seed dormancy and/or increased sensitivity to ABA. The ERF proteins, or a homolog or species ortholog thereof, may be stabilised by mutating the MC-motif at the N-terminal end of the protein. Preferably in such seeds or plants the stabilised ERF proteins, or homolog or species ortholog thereof, accumulate in the cells. Expression of the stabilised ERF proteins, or homolog or species ortholog thereof, may be driven by promoters specific to later seed development, this may help to prevent seed germination while the seed is still on the plant.

According to yet another aspect the invention provides a seed or plant in which expression of one or more of the ERF proteins, or homolog or species ortholog thereof, has been significantly reduced or removed and the seed or plant displays decreased seed dormancy and/or decreased sensitivity to ABA. The ERF proteins, or homolog or species ortholog thereof, may be deleted by mutating the endogenous gene or genes such that no, or a reduced level, of functional protein is expressed.

The invention further provides a transgenic plant comprising an expression vector comprising a promoter operably linked to a polynucleotide encoding a hypoxia resistant mutant hypoxia related protein, wherein the plant shows higher tolerance for hypoxia than a plant not comprising the expression vector.

The hypoxia related protein may be selected from HRE1, HRE2, RAP2.12, RAP2.2, RAP2.3, and a homolog or species ortholog thereof, in particular the hypoxia related protein may be HRE1 or HRE2, or a homolog or species ortholog thereof

The hypoxia resistant mutant hypoxia related protein may be generated by substituting the cysteine at amino acid position 2 of the hypoxia related protein with another amino acid.

The hypoxia resistant mutant hypoxia related protein may be generated by deleting at least 2 amino acids from the N-terminus of the hypoxia related protein.

The transgenic plant may be selected from rice, wheat, tobacco, corn, and soy.

In an alternative embodiment the invention further provides a transgenic plant comprising an expression vector comprising a promoter operably linked to a polynucleotide encoding a drought resistant mutant drought related protein, wherein the plant shows higher tolerance for drought than a plant not comprising the expression vector.

The drought related protein may be selected from HRE1, HRE2, RAP2.12, RAP2.2, RAP2.3, and a homolog or species ortholog thereof.

The drought resistant mutant drought related protein may be generated by substituting the cysteine at amino acid position 2 of the drought related protein with another amino acid.

The drought resistant mutant drought related protein may be generated by deleting at least 2 amino acids from the N-terminus of the drought related protein.

The transgenic plant may be selected from rice, wheat, tobacco, corn, and soy.

In all aspects of the invention the plant may be a crop. The plant may be selected from rice, wheat, tobacco, corn, and soy.

The invention further provides harvestable parts of a plant according to the invention, preferably wherein the harvestable parts are seeds; and/or products directly derived from said plant; and/or products derived directly from said harvestable parts.

The data presented herein demonstrates that a unique amino acid sequence at the N-terminus of specific mature proteins directs such proteins for degradation, initiating with the amino acids MC-. Manipulating the extent to which these proteins, and in particular hypoxia-related proteins, are directed for degradation allows for generation of plants, in particular crop plants, more resistant to oxygen stress and/or which have improved water use efficiency or improved resistance to abiotic stresses in general, or improved germination performance, and accumulation of such proteins can be manipulated by levels of oxygen and/or nitric oxide.

The skilled man will appreciate that preferred features of any one embodiment and/or aspect of the invention may be applied to all other embodiments and/or aspects of the invention.

There now follows by way of example only a detailed description of the present invention with reference to the accompanying drawings, in which:

### BRIEF DESCRIPTION OF THE DRAWINGS

FIG. 1 Diagrammatic representation of the N-end rule of targeted proteolysis (NERP) (after Graciet and Wellmer 2010⁹).

Tertiary, secondary and primary destabilizing residues are highlighted, referring to the number of steps required before recognition for proteosomal degradation. Primary destabilizing residues associated with PRT6 are boxed in red.

Colored ovals represent protein substrates. Single letter amino acid codes are shown for N-terminal residues. MAP, Methionine Amino Peptidase; C* represents oxidised C; NTAN1 and NTAQ1, Nt-amidases for Asn and Gln; ATE, Argenyl tRNA Transferase; PRT, Proteolysis (E 3 ligase).

FIGS. 2A-2F N-end rule mutants ectopically accumulate anaerobic response mRNAs and are more tolerant to hypoxia.

FIG. 2A Genes differentially regulated comparing WT (Col-0) and mutants under air or hypoxia (2 h —O₂).

FIG. 2B mRNAs upregulated in mutants overlap with 49 mRNAs induced across cell types by hypoxia in WT seedlings¹⁵.

FIG. 2C Spatial visualization of ADH1 promoter activity. Scale bars 100 μm.

FIG. 2D Germination under reduced oxygen availability.

FIG. 2E Seedlings after 12 h of hypoxia and 3 d recovery. Scale bar 0.6 cm.

FIG. 2F NERP mutants are less sensitive to hypoxia stress. Data are mean of replicate experiments±SD; *=P<0.05, **=P<0.01.

FIGS. 3A-3B Time course analysis of pADH1::GUS expression in Col-0 (WT) and prt6 mutant 7 day old seedlings. Time (h) after transfer to hypoxic conditions is indicated for each sample.

FIG. 3A Imaging of whole seedlings

FIG. 3B Imaging of the root/shoot junction and seed coat. Bar indicates 100 μm

FIGS. 4A-4D Group VII ERF transcription factors are substrates for the NERP in vitro and in vivo.

FIG. 4A In vitro stability of HA-tagged WT and C2A variants of Arabidopsis Group VII ERFs in the absence or presence of MG132, NERP competitive dipeptides (Arg-Ala) or non competitive dipeptides (Ala-Ala).

FIG. 4B In vitro stability of WT and C2A VRN2-HA and MAF5-HA.

FIG. 4C In vitro stability of HA-tagged rice ERFs.

FIG. 4D shows measured in vivo longevity of the ERF proteins HRE1 and 2.

FIGS. 5A-5B Arabidopsis and rice ethylene responsive factor proteins with a MC-N terminus.

FIG. 5A ClustalW³⁷ alignment of Arabidopsis (At) and rice (Os) ERFs with a N-terminal MC-dipeptide. ERF group assignments are from Nakano et al¹⁷. ERF group assignments in red are based on phylogenetic characterization of the SK and SUB 1 genes²¹. Conserved residues are shaded. An aspartic acid (E, red box) of SUB1A-1 was the target of mutation.

FIG. 5B N-terminal sequences of MADS AFFECTING FLOWERING 5 (MAF5) and REDUCED VERNALIZATION RESPONSE 2 (VRN2) that were used as controls in this study.

FIGS. 6A-6E HRE proteins are stabilized under low oxygen and confer hypoxia tolerance.

FIG. 6A In vivo stability of WT and C2A HRE1-HA, HRE2-HA (a-HA) or S6 control (a-S6): no stress (NS), 2 h hypoxia (HS), following 1 h recovery from stress (R).

FIG. 6B Seedlings expressing WT or C2A HRE1-HA and HRE2-HA after 12 h hypoxic stress and 3 d recovery. Scale bar=0.6 cm.

FIG. 6C Seedling survival for WT or C2A HRE1-HA and HRE2-HA after 9 h or 12 h hypoxic stress. Data are mean of replicate experiments+/−SD; *=P<0.05; **=P<0.01.

FIG. 6D shows seeds and seedlings ectopically expressing stable C2A versions of HRE1 and HRE2 had increased tolerance to extended periods of oxygen deprivation.

FIG. 6E shows a mechanism of stabilization of Arabidopsis ERF Group VII transcription factors under hypoxic conditions leads to increased survival under low oxygen stress.

FIG. 7A-7B HRE proteins are stabilized under low oxygen and improve survival of hypoxia.

FIG. 7A In vivo stability of WT and C2A variants of HRE1-HA and HRE2-HA under non-stress (NS), 2 h hypoxic stress (HS) and after 1 h recovery from 2 h HS (R) in 7-d-old Arabidopsis seedlings. Data for each transgene are shown for two independent lines (1 and 2). Equal amounts of protein were loaded in each gel lane and ribosomal protein S6 (S6) was used as a loading control.

FIG. 7B Survival data of 7-day old seedlings expressing WT or C2A variants of HRE1-HA and HRE2-HA after 9 h or 12 h hypoxic stress. Data for each transgene are shown for two independent lines (1 and 2). Data represent mean±SD from a representative experiment of three biological replicates. An asterisk indicates a significant difference between the transgenic and WT seedlings grown on the same plate (*=P<0.05, **=P<0.01).

FIGS. 8A-8B The N-end rule pathway regulates stomatal aperture responses to ABA. prt6 mutants have ABA hypersensitive stomata. Mean stomatal apertures of wild-type (Col-0), prt6-1 and prt6-5 measured from epidermal strips of mature Arabidopsis leaves, following incubation with concentrations of exogenous ABA indicated (FIG. 8A, left graph). Stomatal aperture values as a percentage of values for the same genotype in the absence of added ABA (FIG. 8B, right panel). prt6 mutant stomata are significantly more closed than those of the wild-type control in the presence of exogenous ABA (p<0.05).

FIG. 9 The N-end rule pathway regulates stomatal frequency. Abaxial prt6 stomatal indices are significantly lower than Col-0 wild-type (p<0.05).

FIG. 10 The N-end rule pathway regulates plant water loss. Relative water content of prt6 plants is higher than controls under normal watering conditions or during water stress. RWC of control (Col-0), prt6-1 and prt6-5 leaves under normal watering conditions or after one week drought with no water supplied. Values are means of 4 measurements of 10 leaves from 10 plants. prt6 mutant plants have significantly higher RWC than wild-type control under drought conditions (p<0.05).

FIG. 11 The N-end rule pathway regulates leaf transpiration. Infrared thermal imaging reveals higher temperature of prt6 mutants under drought conditions indicating reduced evapotranspiration in comparison to col-0 wild-type controls. False colour temperature scale shown on right.

FIG. 12 The N-end rule pathway influences plant drought tolerance. Infra-red thermal images and photographs of 6 week old wild-type (Col-0) and prt6-5 plants. Plants were subjected to drought for and then re-watered for 3 days. False colour temperature scale shown on right.

FIG. 13A The N-end rule pathway controls seed dormancy through MC-initiating proteins that act as sensors for Nitric Oxide (NO) levels. Whereas in Col-0 WT seeds dormancy can be broken by SNP (a donor of NO), this is not the case in prt6, ate1ate2 or maplA seeds treated with fumagillin (that inhibits all MAP activity).

FIG. 13B Proteins initiating MC control seed sensitivity to ABA. Seeds of the maplA mutant germinated in the presence of fumagillin (that inhibits MAP2 activity) are hypersensitive to ABA, whereas WT Col-0 seeds are not.

FIG. 13C Triple mutant seeds in which RAP2.12, RAP2.2 and EBP activities are removed in the presence of the prt6 mutation show insensitivity to ABA. rap2.12 rap2.2 prt6 or rap2.12 ebp prt6 triple mutants show reduced sensitivity to ABA compared to WT Col-0 and the ABA hypersensitive mutant prt6. This shows that hypersensitivity of prt6 works through the stabilisation of MC-ERFs, and removal of the substrates genetically removes ABA sensitivity, showing that these are the substrates controlling ABA sensitivity of germination.

FIG. 14A The reporter MC-GUS is an artificial N-end rule substrate in-vitro. Both MC and MA-GUS proteins were produced in a rabbit reticulocyte lysate that contains all the necessary components of the N-end rule pathway. Where as MC-GUS was degraded within 120 minutes (and not degraded in the presence of the proteasome inhibitor MG132 and competitive inhibits of the N-end rule pathway Arg-Ala), MA-GUS was not.

FIG. 14B Transgenic plants were produced containing MC- or MA-GUS in either WT or prt6 mutant backgrounds (constitutive gene transcription produced from the 35S CaMV promoter). Whereas MA-GUS is stable in WT Col-0 seedlings (as observed by blue staining), MC-GUS was only stabilised in the prt6 background (only blue staining was observed in prt6, not in WT Col-0 seedlings), demonstrating that it is a substrate in vivo.

FIG. 14C Stability of MC-GUS in Col-0 is related to NO. Western analysis of MC-GUS protein levels in WT Col-0 seedlings treated with cPTIO (a scavenger of NO) show that under these conditions MC-GUS protein accumulates. This demonstrates that NO controls the stability of MC-initiating proteins in plants.

FIG. 14D Stability of the physiological substrate HRE2 is controlled by NO. Western blot showing levels of HRE2 protein in-vivo (constitutive gene transcription produced from the 35S CaMV promoter). Treatment with cPTIO stabilises the protein, showing that NO is required for degradation.

### DETAILED DESCRIPTION OF THE INVENTION

Provided herein are nucleic acids and expression vectors encoding hypoxia resistant hypoxia-related proteins that are controlled by NERP (N-end rule proteolysis). Hypoxia related proteins that are controlled by NERP include HRE1, HRE2, RAP2.12, RAP2.2, RAP2.3, allelic variants, homologs, and species orthologs thereof. Such hypoxia related proteins are rendered hypoxia resistant by replacing the cysteine at position 2 with another amino acid that is not oxidized according to the NERP pathway. Further provided are transgenic plants comprising such expression vectors.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

A polynucleotide “exogenous” to an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g., in Arabidopsis by vacuum infiltration) or R₀ (for plants regenerated from transformed cells in vitro) generation transgenic plant.

As used herein, the term “transgenic” describes a non-naturally occurring plant that contains a genome modified by man, wherein the plant includes in its genome an exogenous nucleic acid molecule, which can be derived from the same or a different plant species. The exogenous nucleic acid molecule can be a gene regulatory element such as a promoter, enhancer, or other regulatory element, or can contain a coding sequence, which can be linked to a heterologous gene regulatory element. Transgenic plants that arise from sexual cross or by selfing are descendants of such a plant and are also considered “transgenic.”.

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only “substantially identical” to a sequence of the gene from which it was derived. As explained below, these substantially identical variants are specifically covered by reference to a specific nucleic acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

A “homolog” refers to a gene or protein which is substantially identical to the gene or protein identified or referred to.

A “species ortholog” refers to a gene or protein in another species whose encoded protein or protein fulfills a similar role to the gene or protein identified or referred to. The species ortholog may have some sequence identity with the identified gene or protein. The percent identity may rang from 25% to 100%, for example at least: 25%, 30%, 35%, 40%, 45%, 50%, 55%, 60%, 65%, 70%, 75%, 80%, 85%, 90%, 95%, or 99% sequence identity.

The phrase “substantially identical,” used in the context of two nucleic acids or polypeptides, refers to a sequence that has at least 25% sequence identity with a reference sequence. Alternatively, percent identity can be any integer from 25% to 100%. Some embodiments include at least: 25%, 30%, 35%, 40%, 45%, 50%, 55%, 60%, 65%, 70%, 75%, 80%, 85%, 90%, 95%, or 99%, compared to a reference sequence using the programs described herein; preferably BLAST using standard parameters, as described below. The present invention provides for nucleic acids encoding polypeptides that are substantially identical to hypoxia resistant mutant hypoxia related proteins, e.g., HRE1, HRE2, RAP2.12, RAP2.2, and RAP2.3.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

### Recombinant Expression Vectors

Once the coding or cDNA sequence for a protein, such as hypoxia resistant mutant hypoxia related protein or an MC-motif protein, is obtained, it can also be used to prepare an expression cassette for expressing the protein in a transgenic plant, directed by a heterologous promoter. Increased expression of the related polynucleotide is useful, for example, to produce plants with enhanced drought-resistance or other improved growth characteristics. Alternatively, as described above, expression vectors can also be used to express polynucleotides and variants thereof that inhibit endogenous protein expression.

Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, the hypoxia resistant mutant hypoxia related gene can be expressed constitutively (e.g., using the CaMV 35S promoter).

To use hypoxia resistant mutant hypoxia related coding or cDNA sequences in the above techniques, recombinant DNA vectors suitable for transformation of plant cells are prepared, such vectors can be used to express all mutant proteins within the scope of the invention. Techniques for transforming a wide variety of higher plant species are well known and described in the technical and scientific literature. See, e.g., Weising et al. Ann. Rev. Genet. 22:421-477 (1988). A DNA sequence coding for the hypoxia resistant mutant hypoxia related polypeptide preferably will be combined with transcriptional and translational initiation regulatory sequences which will direct the transcription of the sequence from the gene in the intended tissues of the transformed plant.

For example, a plant promoter fragment may be employed to direct expression of the hypoxia resistant mutant hypoxia related gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the hypoxia resistant mutant hypoxia related protein in a specific tissue (tissue-specific promoters) or may be otherwise under more precise environmental control (inducible promoters). Examples of tissue-specific promoters under developmental control include promoters that initiate transcription only in certain tissues, such as leaves or guard cells (including but not limited to those described in WO/2005/085449; U.S. Pat. No. 6,653,535; Li et al., Sci China C Life Sci. 2005 April; 48(2):181-6; Husebye, et al., Plant Physiol, April 2002, Vol. 128, pp. 1180-1188; and Plesch, et al., Gene, Volume 249, Number 1, 16 May 2000, pp. 83-89(7)). Examples of environmental conditions that may affect transcription by inducible promoters include anaerobic conditions, elevated temperature, or the presence of light.

If proper protein expression is desired, a polyadenylation region at the 3′-end of the coding region should be included. The polyadenylation region can be derived from the natural gene, from a variety of other plant genes, or from T-DNA.

The vector comprising the sequences (e.g., promoters or hypoxia resistant mutant hypoxia related coding regions) will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, the hypoxia resistant mutant hypoxia related nucleic acid sequebce, or indeed any other nuclei acid sequence, is expressed recombinantly in plant cells to enhance and increase levels of total hypoxia resistant mutant hypoxia related polypeptide or of another polypeptide. A variety of different expression constructs, such as expression cassettes and vectors suitable for transformation of plant cells can be prepared. Techniques for transforming a wide variety of higher plant species are well known and described in the technical and scientific literature. See, e.g., Weising et al. Ann. Rev. Genet. 22:421-477 (1988). A DNA sequence coding for a hypoxia resistant mutant hypoxia related protein can be combined with cis-acting (promoter) and trans-acting (enhancer) transcriptional regulatory sequences to direct the timing, tissue type and levels of transcription in the intended tissues of the transformed plant. Translational control elements can also be used.

The invention provides for nucleic acids, such as a hypoxia resistant mutant hypoxia related nucleic acid, operably linked to a promoter which, in some embodiments, is capable of driving the transcription of the protein coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

A. Constitutive Promoters

A promoter fragment can be employed to direct expression of a hypoxia resistant mutant hypoxia related nucleic acid, or indeed any relevant nucleic acid, in all transformed cells or tissues, e.g., as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding a hypoxia resistant mutant hypoxia related protein or another protein according to the invention (Comai et al., Plant Mol. Biol. 15:373 (1990)).

Other examples of constitutive promoters include the 1′- or 2′-promoter derived from T-DNA of Agrobacterium tumafaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

B. Inducible Promoters

Alternatively, a plant promoter may direct expression of the hypoxia resistant mutant hypoxia related gene or another related gene according to the invention under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention can incorporate drought-specific promoter such as the drought-inducible promoter of maize (Busk (1997) supra); or alternatively the cold, drought, and high salt inducible promoter from potato (Kirch (1997) Plant Mol. Biol. 33:897-909).

Alternatively, plant promoters which are inducible upon exposure to plant hormones, such as auxins, are used to express the hypoxia resistant mutant hypoxia related gene or another gene according to the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

Plant promoters inducible upon exposure to chemicals reagents that may be applied to the plant, such as herbicides or antibiotics, are also useful for expressing the hypoxia resistant mutant hypoxia related gene or another gene according to the invention. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. A desired coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Examples of useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

C. Tissue-Specific Promoters

Alternatively, the plant promoter may direct expression of the hypoxia resistant mutant hypoxia related gene, or another gene according to the invention, in a specific tissue (tissue-specific promoters) manner. Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue, or epidermis or mesophyll. Reproductive tissue-specific promoters may be, e.g., ovule-specific, embryo-specific, endosperm-specific, integument-specific, seed and seed coat-specific, pollen-specific, petal-specific, sepal-specific, or some combination thereof. In some embodiments, the promoter is cell-type specific, e.g., guard cell-specific.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmyc1 from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express polynucleotides encoding hypoxia resistant mutant hypoxia related polypeptides (or RNAi or antisense constructs specific for NERP gene products). For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes that exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are knl-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the hypoxia resistant mutant hypoxia related polynucleotide, or another polynucleotide according to the invention, is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

### Production of Transgenic Plants

As detailed herein, the present invention provides for transgenic plants comprising recombinant expression cassettes encoding hypoxia resistant mutant hypoxia related proteins, or other proteins according to the invention, in a plant or for inhibiting or reducing endogenous NERP activity. Thus, in some embodiments, a transgenic plant is generated that contains a complete or partial sequence of an endogenous hypoxia resistant mutant hypoxia related protein encoding polynucleotide, or another related polynucleotide. In some embodiments, a transgenic plant is generated that contains a complete or partial sequence of a polynucleotide that is from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a hypoxia resistant mutant hypoxia related coding sequence, or coding sequence encoding another protein relevant to the invention, driven by a heterologous promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of hypoxia resistant mutant hypoxia related is encompassed by the invention, generally expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Transformed plant cells derived by any of the above transformation techniques can be cultured to regenerate a whole plant that possesses the transformed genotype and thus the desired phenotype such as enhanced drought-resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes of the invention can be used to confer drought resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is selected from the group consisting of rice, maize, wheat, soybeans, cotton, canola, turfgrass, and alfalfa. In some embodiments, the plant is an ornamental plant. In some embodiment, the plant is a vegetable- or fruit-producing plant.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

The plants of the invention have enhanced resistance to hypoxia compared to wild type plants. Hypoxia resistance can assayed according to any of a number of well-known techniques. For example, plants can be grown under conditions in which less than optimum oxygen is provided to the plant (e.g., flooded or submerged). Hypoxia resistance can be determined by any of a number of standard measures including turgor pressure, growth, yield, and the like. In some embodiments, the methods described in the Example section, below can be conveniently used.

### EXAMPLES

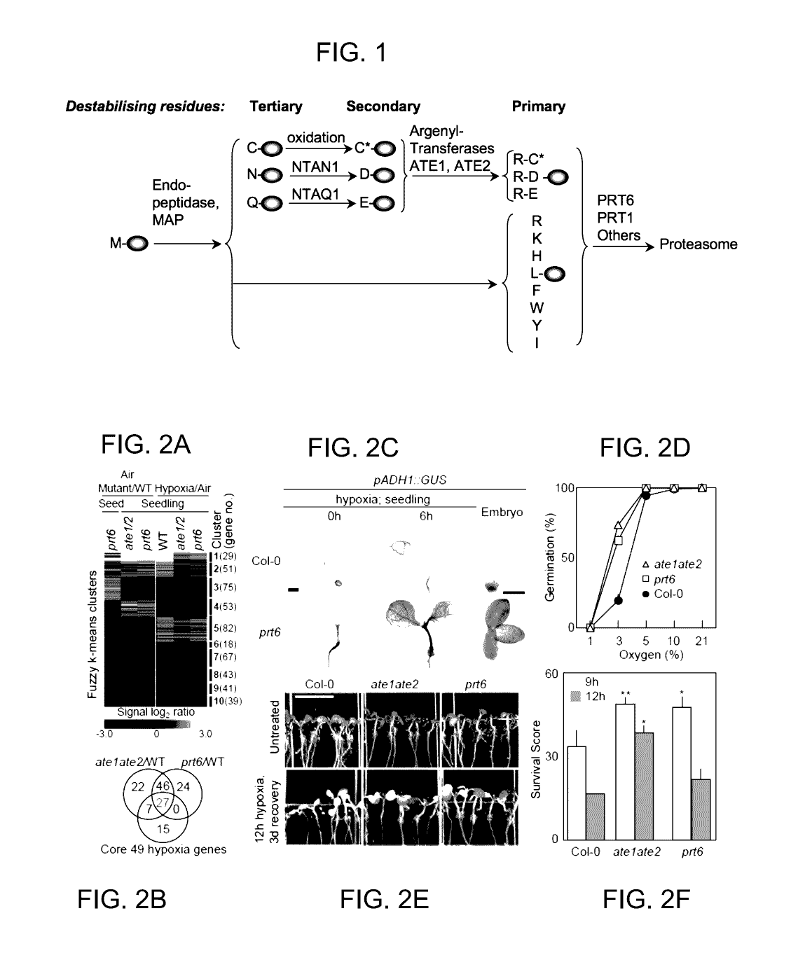

The N-end rule pathway of targeted proteolysis (NERP) associates the fate of a protein substrate with the identity of its N-terminus (the N-degron)^{5,6}. The N-terminal residue is classified as stabilizing or destabilizing, depending on the fate of the protein. An N-degron containing a destabilizing residue is created through specific proteolytic cleavage, but can also be generated via successive enzymatic or chemical modifications to the N-terminus, for example, arginylation by Arg-tRNA protein transferases (ATE)7,8,9 (FIG. 1). NERP substrates containing destabilizing residues are targeted for proteasomal degradation via specific E3 ligases (also known as N-recognins), such as PROTEOLYSIS1 and 6 (PRT1 and 6) in Arabidopsis, which accept substrates with hydrophobic and basic N-degrons, respectively^{8,9,10}. Several substrates of the NERP are important developmental regulators in mammals¹¹ but as yet no substrates have been identified in plants. Previously we showed a function of this pathway in Abscisic Acid (ABA) signaling through PRT6 and ATE¹², and it has also been associated with leaf senescence and shoot and leaf development^{13,14} in Arabidopsis.

### Methods

Protein stability analyses. Full length cDNAs were PCR amplified from either Arabidopsis thaliana or Oryza sativa L. (cv. M202 (Sub1)). N-terminal mutations were introduced using a specific forward primer. For in vitro assays, cDNAs were cloned into a modified version of the pTNT vector (Promega) to produce C-terminal HA fusions. Stability assays were performed using the TNT T7 Coupled Reticulocyte Lysate system (Promega), essentially as described previously²³. For in vivo analysis of EIRE-HA proteins, cDNAs were cloned into pE2c, mobilized into pB2GW7 and transformed into Arabidopsis using the floral dip method. To Assess relative protein stability, equal amounts of total protein extracted from 7-day old T3 homozygous seedlings were analyzed by Western blot, and cDNA synthesized from total RNA was used as a template for semi-quantitative PCR.

Gene expression analyses. For microarray analysis, total RNA extracted from seeds¹² or seedlings¹⁵ was hybridized against the Arabidopsis ATH1 genome array (Affymetrix). Differentially expressed genes were clustered as described previouslyl⁵. pADH::GUS¹⁶ was crossed to prt6-1 and homozygous seeds or seedlings were analyzed for GUS activity before and after submergence for the times indicated.

Low O₂ phenotypic analyses. To assess germination (scored as radicle emergence); imbibed seeds were incubated for 7-days in chambers flushed with varying O₂ tensions²⁹. For 7-day old seedling survival, O₂ deprivation was achieved bubbling 99.995% Argon through water into chambers under positive pressure, before recovering in air for 3 days and assessing survival as described previouslyl⁵. The same argon chambers were used to treat seedlings for the times indicated prior to protein extraction for Western blot analysis. Growth and analysis of plant material: Arabidopsis thaliana seed were obtained from NASC, except for transgenics containing pADH::GUS16 (a gift from Robert Ferl; University of Florida, Gainesville). Columbia-0 (Col-0) was the wild type for all analyses. prt6-1, prt6-5 and ate1 ate2 mutants were described previouslyl^{2,14}. For the generation of transgenic Arabidopsis and in vivo protein assays, plants were grown vertically on ½ MS for seven days at 22° C. in 150 μmol m⁻² s⁻¹ constant light and transferred to soil after two weeks if required. For analysis of seedling O2 deprivation survival and protein analysis, plants were grown vertically on MS medium (0.43% (w/v) MS salts, 1% (w/v) Suc and 0.4% (w/v) phytagel, pH 5.75) at 23° C. with a 16-h-day (50 μmol m⁻² s⁻¹) and 8-h-night cycle for 7 d. The rice (Oryza sativa L.) Sub1 introgression line cv. M202(Sub1) was grown and submerged prior to cDNA isolation as described previously′.

Analysis of oxygen deprivation response in seeds and seedlings: Seven day old Arabidopsis seedlings were subjected for specified durations to nonstress (NS) or hypoxia stress (HS) treatments, or subjected to hypoxia stress and returned to ambient air (reoxygenation; R). For seedling survival, 15 Col-0 and 15 mutant seedlings were grown side by side (3 replicates). Treatments commenced at the end of the 16 h light cycle in open (NS) or sealed (HS) chambers. For HS, 99.995% Argon gas was bubbled through water and into the chamber while air was expelled by positive pressure³⁰. After treatment, non-damaged, damaged and dead seedlings were scored as described previously³¹, or seedlings were frozen under liquid nitrogen within 3 min of release prior to protein extraction. Germination of Arabidopsis seeds (3-4 reps of n=600-100; scored on day 7 as radicle emergence) was performed at 22° C. under constant light in various oxygen tensions achieved through mixing N2 and air via capillary tubes according to the apparatus described previously²⁹. Wild type plants carrying the pADH::GUS transgene¹⁶ were crossed to prt6-1 plants and homozygous prt6-1 pADH::GUS individuals were identified in the F2 population. Seven day-old seedlings were submerged in degassed water in the dark to induce hypoxia for the times indicated. Embryos were dissected 6 hours after imbibition. Seedlings and embryos were assayed for GUS activity and imaged following standard methods³².

Construction of transgenic plants and protein and RNA extractions: To generate C-terminally HA-tagged ERF fusions of HRE1 (At1g72360) and HRE2 (At2g47520) driven by the 35SCaMV promoter, full length cDNAs amplified from Arabidopsis total seedling cDNA were first ligated into the Entry vector pE2c and then mobilized into the Destination binary vector pB2GW7, as described previously³³. N-terminal mutations were incorporated by changing the forward primer sequences accordingly (Table 1). Transformation into Agrobacterium tumefaciens (strain GV3101 pMP90) and Arabidopsis thaliana was performed according to established protocols³⁴. Proteins were extracted from 7-day-old homozygous T3 seedlings as described³⁵. Extracts were quantified using the Bio-Rad DC assay and subjected to anti-HA immunoblot analysis. For semi-quantitative RT-PCR, RNA was extracted from using an RNEasy plant mini kit (Qiagen) and converted to cDNA using Superscript III Reverse transcriptase (Invitrogen). PCRs were performed with transgene specific primers (gene specific forward, HA-tag reverse) and Actin-2 was amplified for use as a loading control (Table 1).

In vitro analysis of protein stability: To generate Arabidopsis and rice protein-HA fusions driven by the T7 promoter, cDNAs were PCR amplified from Arabidopsis total cDNA or submerged rice cDNA (M202 (Sub1); as described³ and ligated into a modified version of the pTNT (Invitrogen) expression vector (pTNT3xHA). N-terminal mutations were incorporated by changing the forward primer sequences accordingly (Table 1). Proteins were expressed in vitro using the TNT T7 Coupled Reticulocyte Lysate system (Promega) according to manufacturer's guidelines, using 500 ng plasmid template. Where appropriate, 100 μM MG132 or 1 mM dipeptides (arg-β-ala or ala-ala; Sigma-Aldrich) and 150 nm Bestatin (Sigma-Aldrich) were added. Reactions were incubated at 30° C., and samples were taken at appropriate time points before mixing with protein loading dye to terminate protein synthesis. Equal amounts of each reaction were subjected to anti-HA immunoblot analysis.

Immunoblotting: Proteins resolved by SDS-PAGE were transferred to PVDF using a MiniTrans-Blot electrophoretic transfer cell (Bio-Rad). Membranes were probed with primary antibodies at the following titres: anti-HA (Sigma-Aldrich), 1:1000; anti-α-tubulin (Sigma-Aldrich), 1:5,000, or anti-ribosomal protein 5636, (1:5,000). HRP conjugated anti-mouse secondary antibody (Santa Cruz) was used at a titre of 1:10,000. Immunoblots were developed to film using ECL Western blotting substrate (Pierce). Alignment of MC-ERF proteins from Arabidopsis and Rice: Rice and Arabidopsis ERF proteins starting with the sequence MC- were aligned and phylogenetic relationships observed using CLUSTALW37.

Microarray hybridization and data analyses: Total RNA extracted from seeds or seedlings was assessed for quality using the Agilent 2100 Bioanalyzer with the RNA 6000 Nano reagent kit. Biotin-labelled cRNA was synthesised using the Affymetrix 3′ IVT Express Labelling kit and hybridized against the Arabidopsis ATH1 genome array (GeneChip System®, Affymetrix). CEL file data were processed to estimate the abundance of each expressed mRNA in two (seedling) or three (imbibed seed) biological replicate samples as described previously¹⁵. The microarray experiments reported here are described following MIAME guidelines and deposited in GEO under the accession number (submitted, to be confirmed). The differentially expressed genes were further analyzed by use of fuzzy k means clustering with the FANNY function from the Cluster package in R, as described¹⁵. The resulting gene-to-cluster assignments were visualized with the TIGR MEV program. Each gene cluster was evaluated for enrichment of specific gene functions (Gene Ontology, GO) as described previously³⁸ using Arabidopsis gene-to-GO mappings from TAIR (http://geneontology.org; downloaded May 17, 2011).

TABLE 1

| Primer name | Gene ID | Sequence |
|---|---|---|
| Actin2_F | At3g18780 | 5′-atggctgaggctgatg |
| | | atattc-3′ |
| Actin2_R | At3g18780 | 5′-agaaacattttctgtg |
| | | aacgattc-3′ |
| 3xHA_R | n/a | 5′-agagtactgctagcgg |
| | | ctta-3′ |
| MAF5_MC_F | At5g65080 | 5′-aaaaacgcgtatgtgt |
| | | cggaagagtgaagc-3′ |
| MAF5_MA_F | At5g65080 | 5′-aaaaacgcgtatggct |
| | | cggaagagtgaagc-3′ |
| MAF5_R | At5g65080 | 5′-TTACTTGAGAAGCGGG |
| | | AGAG-3′ |
| VRN2_MC_F | At4g16845 | 5′-aaaaacgcgtatgtgt |
| | | aggcagaattgtcgc-3′ |
| VRN2_MA_F | At4g16845 | 5′-aaaaacgcgtatggct |
| | | aggcagaattgtcgc-3′ |
| VRN2_R | At4g16845 | 5′-TTACTTGTCTCTGCTG |
| | | TTATTG-3′ |
| HRE1_MC_F | At1g72360 | 5′-aaaggatccatgtgcg |
| | | gaggagctgtaat-3′ |
| HRE1_MA_F | At1g72360 | 5′-aaaggatccatggccg |
| | | gaggagctgtaat-3′ |
| HRE1_R | At1g72360 | 5′-aaatctagatcaggac |
| | | catagacccatgt-3′ |
| HRE2_MC_F | At2g47520 | 5′-aaaggatccatgtgtg |
| | | ggggagctatcat-3′ |
| HRE2_MA_F | At2g47520 | 5′-aaaggatccatggctg |
| | | ggggagctatcat-3′ |
| HRE2_R | At2g47520 | 5′-aaatctagattaattg |
| | | gagtcttgatagctc-3′ |
| RAP2.12_MC_F | At1g53910 | 5′-aaaggatccatgtgtg |
| | | gaggagctataatatc-3′ |
| RAP2.12_MA_F | At1g53910 | 5′-aaaggatccatggctg |
| | | gaggagctataatatc-3′ |
| RAP2.12_R | At1g53910 | 5′-aaatctagatcagaag |
| | | actcctccaatcatg-3′ |
| RAP2.2_MC_F | At3g14230 | 5′-aaaggatccatgtgtg |
| | | gaggagctataatc-3′ |
| RAP2.2_MA_F | At3g14230 | 5′-aaaggatccatggctg |
| | | gaggagctataatc-3′ |
| RAP2.2_R | At3g14230 | 5′-aaaatctagatcaaaa |
| | | gtctccttccagcat-3′ |
| EBP_MC_F | At3g16770 | 5′-aaaggatccatgtgtg |
| | | gcggtgctattatt-3′ |
| EBP_MA_F | At3g16770 | 5′-aaaggatccatggctg |
| | | gcggtgctattatt-3′ |
| EBP_R | At3g16770 | 5′-aaatctagattactca |
| | | tacgacgcaatgac-3′ |
| SUB1A_MC_F | | 5′-AAAAGAATTCATGTGT |
| | | GGAGGAGAAGTGATC-3′ |
| SUB1A_MA_F | | 5′-AAAAGAATTCATGGCT |
| | | GGAGGAGAAGTGATC-3′ |
| SUB1A_E5A_F | | 5′-AAAAGAATTCATGTGT |
| | | GGAGGAGCTGTGATC-3′ |
| SUB1A_R | | 5′-AAAAGGTACCCCCTGC |
| | | ATATGATAT-3′ |
| SUB1C_F | | 5′-AAAAGAATTCATGCGC |
| | | CGCCGCGTCTCC-3′ |
| SUB1C_R | | 5′-AAAAGGTACCGCTCCA |
| | | GAAGCGCATGTCG-3′ |

### Example 1

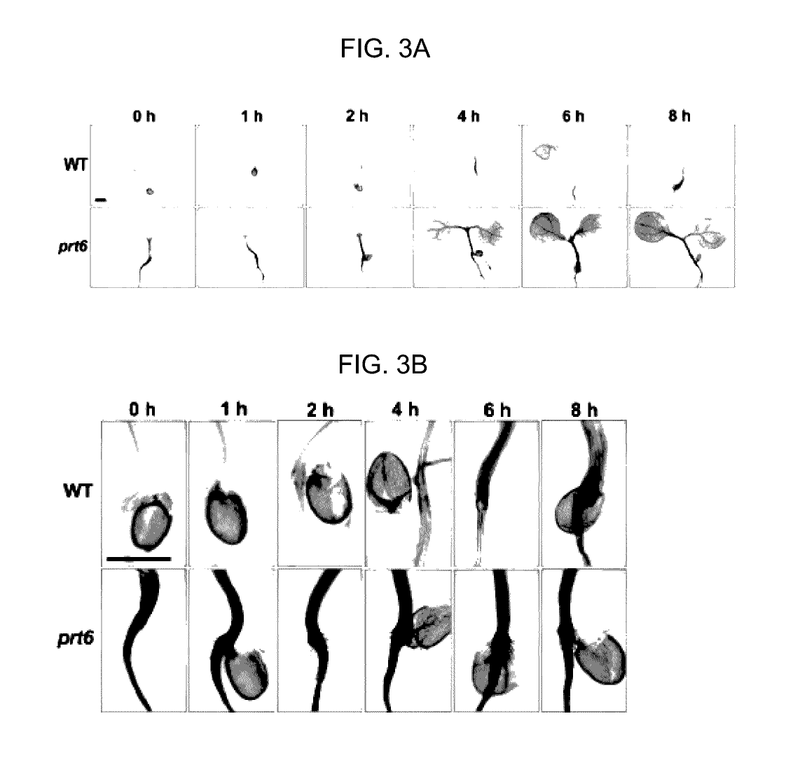

To understand NERP-regulated gene expression we analyzed the transcriptome of imbibed seed and seedling NERP mutants prt6 and ate1 ate2, which lacks ATE activity¹⁴ ((FIG. 1) 1). This analysis revealed that genes important for anaerobic metabolism and survival of hypoxia, such as ALCOHOL DEHYDROGENASE1 (ADH1), were constitutively expressed at high levels in both mutants, in common with wild-type (WT) Col-0 plants under hypoxia. For example, 47 of the 135 differentially regulated mRNAs in the WT hypoxia-induced transcriptome were similarly up-regulated in prt6 seedlings grown under non-stress conditions. The ptr6 and ate1 ate2 up-regulated mRNAs included over half of the core 49 mRNAs up-regulated by hypoxia across seedling cell types¹⁵ ((FIG. 2B) 2b). Consistent with this observation β-glucuronidase (GUS) expression driven by the promoter of ADH1 (pADH1::GUS¹⁶) was upregulated in WT seedlings subjected to hypoxia and ectopically expressed in mature embryos, roots and lower hypocotyls of prt6 (FIG. 2C, FIG. 3). Constitutive expression of hypoxia-induced genes by NERP mutant seedlings suggested that they would be resistant to hypoxic conditions. Imbibed seeds of both prt6 and ate1 ate2 were able to germinate well under low oxygen (3%) compared to WT (FIG. 2d FIG. 2D), and mutant seedlings were more able to survive prolonged oxygen deprivation (FIG. 2c,f FIGS. 2E, 2F).

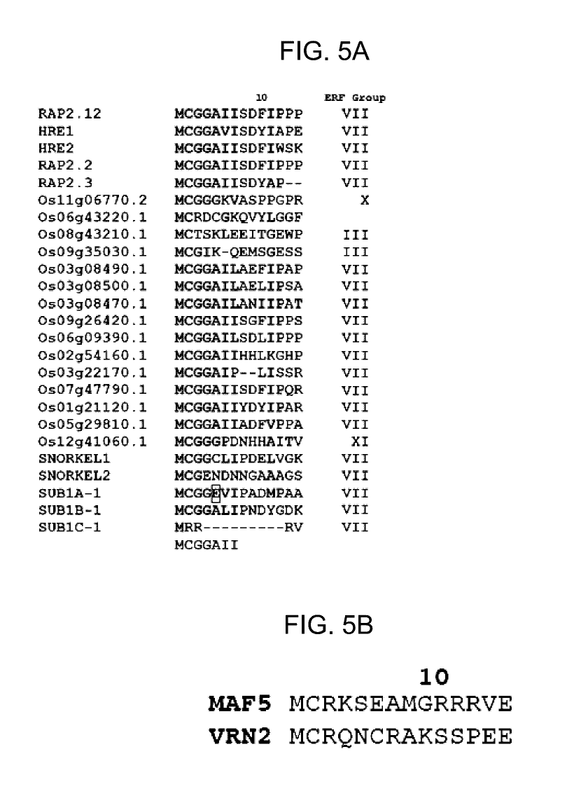

Transcription factors of the five member Arabidopsis Ethylene Response Factor (ERF) Group VII¹⁷ have recently been shown to enhance plant responses to hypoxia or anoxia, including HYPDXIA RESPONSIVE(HRE)1 and 218 and RELATED TO AP2(RAP) RAP2.2¹⁹. Over-expression of RAP2.12 was also shown to induce expression of a pADH1:LUCIFERASE reporter gene²⁰. This subfamily shows homology to the agronomically important rice ERFs SUBMERGENCE(SUB)1A, B, C³ and SNORKEL1 and 2²¹. SUBIA-1 within the SUBMERGENCE1 (Sub1) locus (which also contains SUB1B and SUB1C) was shown to be a primary determinant of enhanced survival of rice plants under complete submergence³. With the exception of SUB1C all contain the motif MC- at the N-terminus, embedded within a longer consensus shared with most other Group VII ERFs of Arabidopsis and rice, MCGGAII (FIG. 5A).

### Example 2

Removal of N-terminal methionine by METHIONINE AMINO-PEPTIDASE(MAP) reveals the tertiary destabilizing residue cysteine in proteins initiating with MC-, which targets substrates for degradation by the NERP7.9.22 (FIG. 1). In mouse, NERP-mediated degradation of the MC-motif containing G-protein signaling components RGS4 and RGS5 is perturbed under hypoxia^{22,23}. It was hypothesized that oxidation of C2 in these proteins under normoxia creates a secondary destabilizing residue allowing addition of R to the N-terminus by ATE, creating a primary destabilizing residue²³.

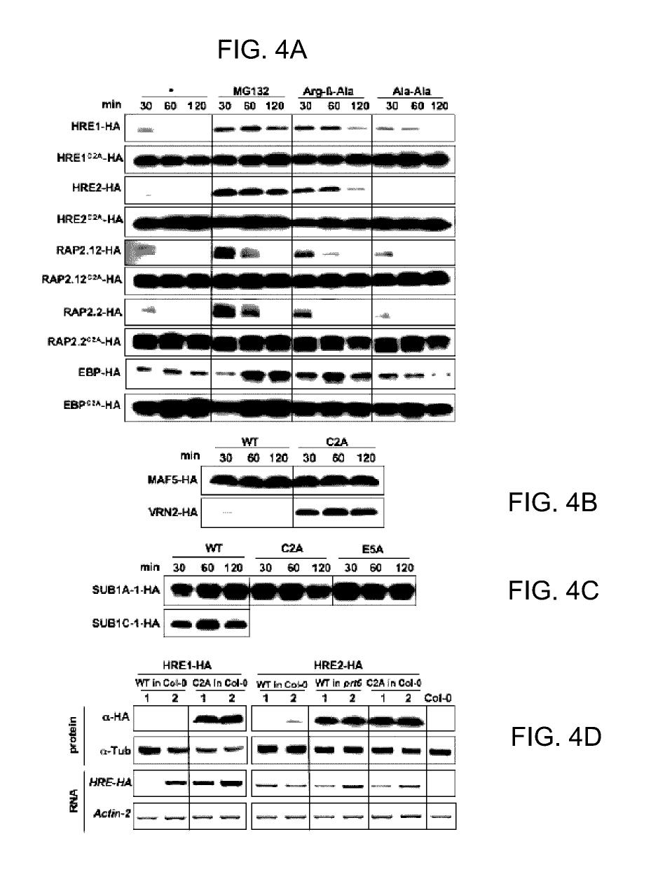

We investigated the possibility that all Arabidopsis Group VII ERFs as well as rice SUB1A-1 were NERP substrates. A heterologous rabbit reticulocyte lysate assay²³ was used, because components of the NERP (ATE, MAP and PRT6) are highly conserved in eukaryotes⁸, and it has been shown that wheat-germ lysate does not contain an active proteosomal system²⁴. Arabidopsis Group VII ERFs were all short-lived, and their stability was enhanced by MG132, and the NERP competitive dipeptide Arg-Ala but not by the noncompetitive Ala-Ala dipeptide²³ (FIG. 4a). Mutation of C2 to A (^{C2A}), which should remove the N-degron and stabilise proteins specifically with respect to the NERP²³, significantly enhanced stability in vitro of Arabidopsis ERFs, indicating that all Group VII ERFs are substrates of the NERP.

Arabidopsis contains 206 proteins from gene models with MC- at the N-terminus; we used two of these, VERNALISATION (VRN)2 and MADS AFFECTING FLOWERING(MAF)5, which lack the extended N-terminal Group VII ERF consensus (FIG. 5B), to test specificity of this sequence. Whereas VRN2-HA was degraded in this system, and stabilized by the introduction of a C2A mutation (VRN2^{c2A}-HA), MAF5-HA and MAF5^{C2A}-HA were both stable (FIG. 4B), indicating that not all Arabidopsis MC-proteins are NERP substrates. This is not surprising as it has previously been shown that optimal positioning of a downstream lysine for ubiquitination is also a key determinant of the quality of an N-degron^{8,9,25}. SUB1A-1 was resistant to degradation (FIG. 4C). As the N-terminal sequence of SUB1A-1 differs at position 5 (E rather than A, FIG. 5A) we analysed a mutant version that replaced this amino acid to reconstitute the consensus Group VII sequence (Sub1A^{E5A}). Sub1A^{E5A} was also stable in vitro (FIG. 4C), suggesting that degradation of this protein is uncoupled from the NERP. As expected, the rice protein SUB1C, lacking an MC N-terminus, was long lived in vitro (FIG. 4C).

To confirm activity of the NERP towards specific MC-containing substrates in plants, we analyzed the in vivo longevity of the ERF proteins HRE1 and 2 (FIG. 4D). We expressed either WT or mutant (HRE1^{c2A}, HRE2^{c2A}) HA-tagged versions of these proteins ectopically using the CaMV35S promoter in Arabidopsis. In WT plants, only the mutant C2A proteins could be detected at high levels, despite detectable expression of corresponding mRNAs, suggesting that WT versions are NERP substrates in vivo. HRE2-HA expressed in the prt6 mutant was stable, linking its degradation directly to PRT6.

### Example 3

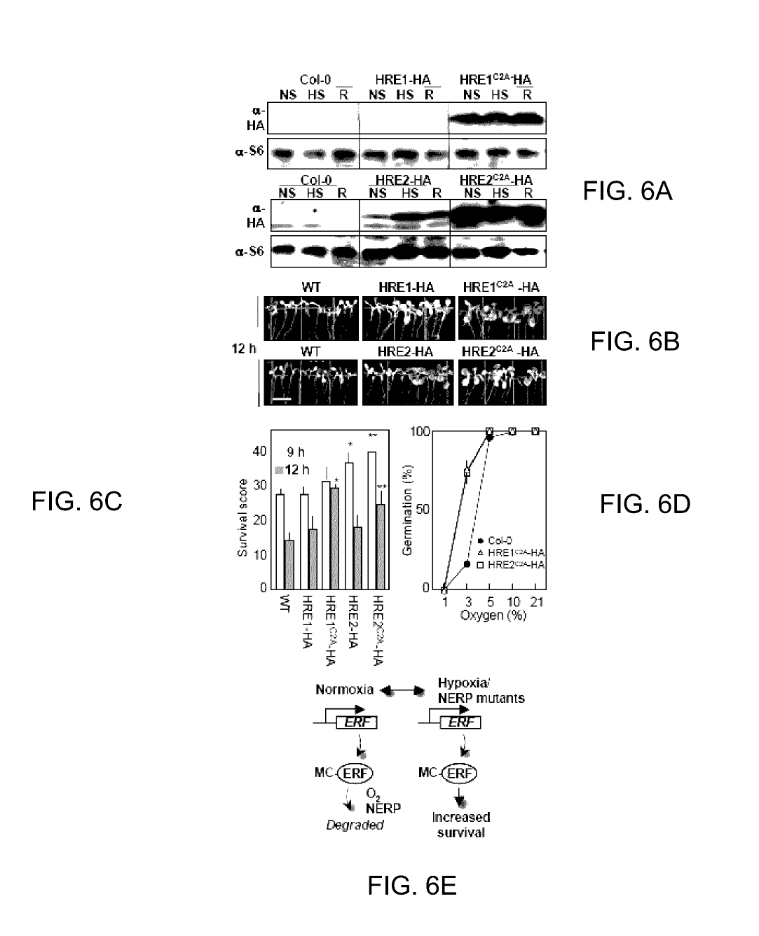

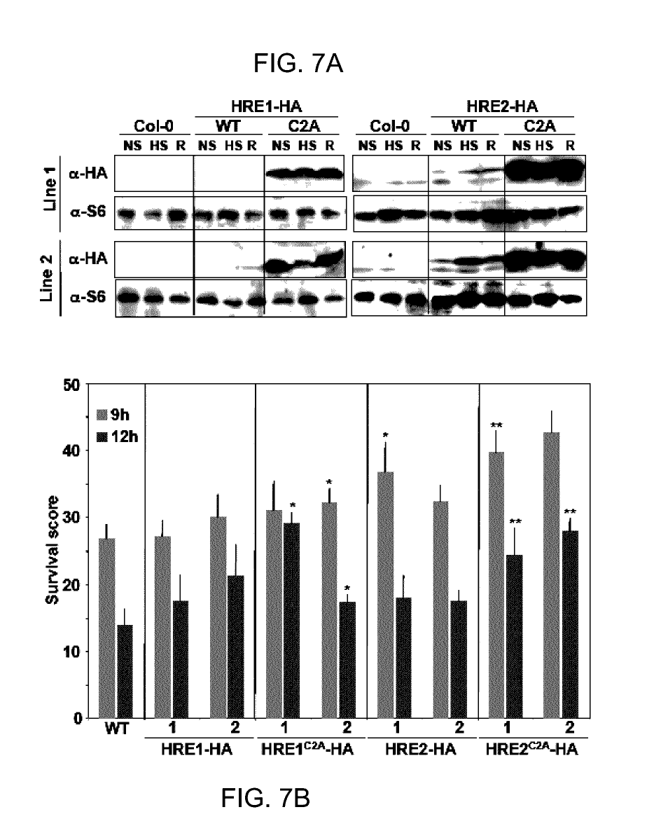

To assess whether oxygen regulates the stability of HRE proteins, we analyzed the accumulation of HRE-HA proteins in WT plants expressing HRE1-HA, HRE1^{C2A}-HA, HRE2-HA and HRE2^{c2A}-HA under normal and low oxygen conditions (FIG. 6A). Following transfer of seedlings to hypoxic conditions we observed elevation of HRE2-HA within 2 hours, but could not detect HRE1-HA (FIG. 6A; FIG. 7A). HRE2-HA, became destabilized again upon return to normoxic conditions (FIG. 6A). Both seeds and seedlings ectopically expressing stable C2A versions of HRE1 and HRE2 had increased tolerance to extended periods of oxygen deprivation (FIGS. 6B, 6C, 6D; FIG. 7B).

These data demonstrate that Arabidopsis ERF Group VII transcription factors are substrates of the NERP, and function to sense molecular oxygen, most likely through oxidation of the tertiary destabilizing residue cysteine. Stabilization of these proteins under hypoxic conditions leads to increased survival under low oxygen stress (FIG. 6E). SUB1A-1 may provide enhanced responsiveness to submergence and drought in rice in part due to the fact that it is not a substrate of the NERP. SUB1A-1 likely evades NERP due to the absence of an optimally positioned lysine downstream of the N-degron, since substrate quality is determined combinatorially by N-degron destabilizing residue and downstream lysine position^{8,9,25}.

Targeted degradation of proteins by the NERP was identified as a homeostatic mechanism in mammalian systems^{22,23,28}, for example in the control of hypoxia-related expression of RGS4²⁸ and RGS5²³. It is fascinating that the NERP carries out the same functionality in relation to low oxygen stress in plants, but taking as substrates members of a plant specific transcription factor family. This highlights evolutionary conservation of the mechanism of oxygen perception across kingdoms using the NERD independent of the targets. The present showing of in vivo function of two members of the Group VII sub-family provides direct evidence for the control of HRE2 by oxygen and the NERP and indirect evidence that HRE1 is also a NERP substrate in vivo. We demonstrate that all members of Arabidopsis Group VII ERFs are NERP substrates in vitro, and thus it is possible that all members orchestrate NERP-controlled, hypoxia-related functions. Identification and manipulation of NERP substrates will therefore be a key target for both conventional breeding and biotechnological approaches in relation to manipulation of plant responses to abiotic stress.

### Example 4

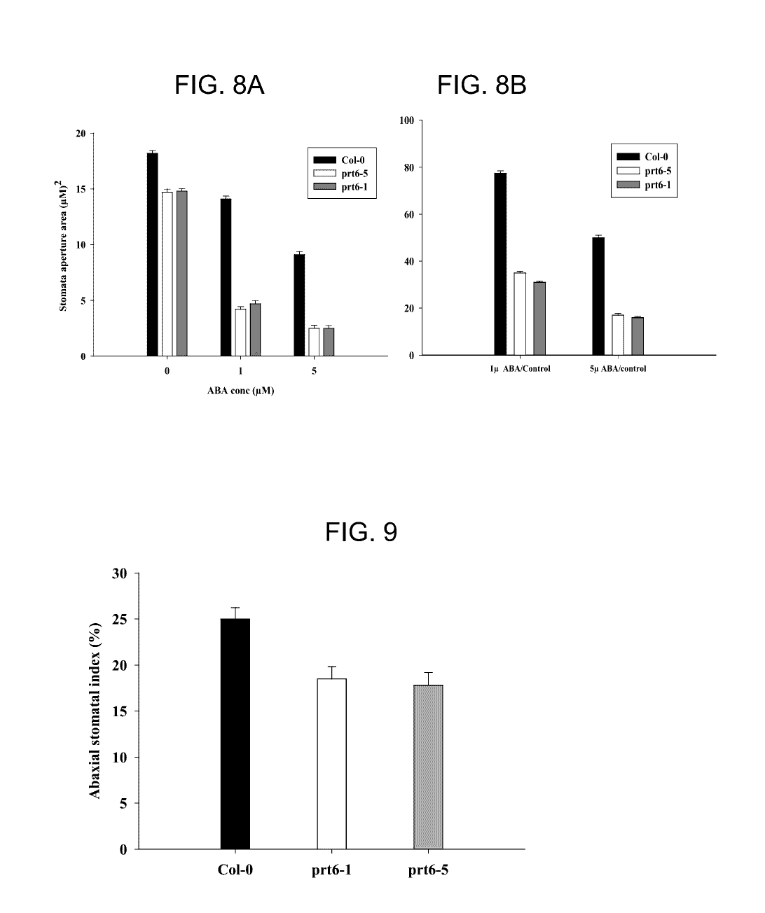

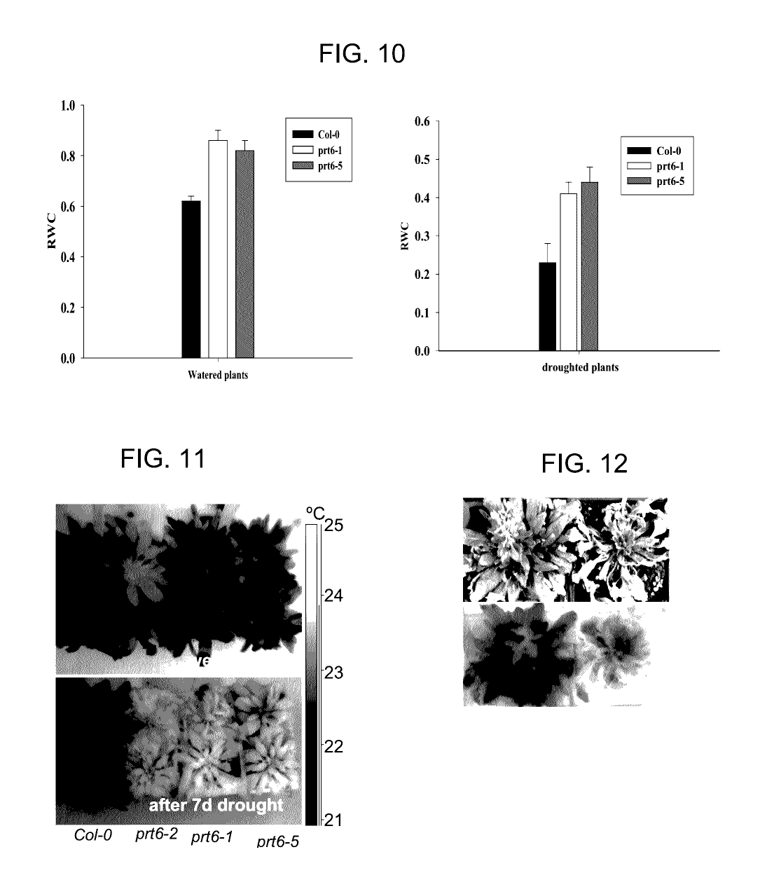

The data presented in FIGS. 8 to 12 demonstrates that by controlling the level of ERF protein in a plant cell improved water use efficiency and/or improved resistance to abiotic stress can be observed. The data shows that prt6-1 and prt6-5 mutant plants, that is plants that are unable to degrade MC-motif proteins via the N-end rule pathway, display improved water use efficiency and drought tolerance. The prt6-1 and prt6-5 mutant plants are N-end rule pathway mutants with guard cells that are hypersensitive to ABA. The plants also display lower stomatal density and index leading to reduced transpiration and improved drought tolerance when water-stressed.

### Example 5

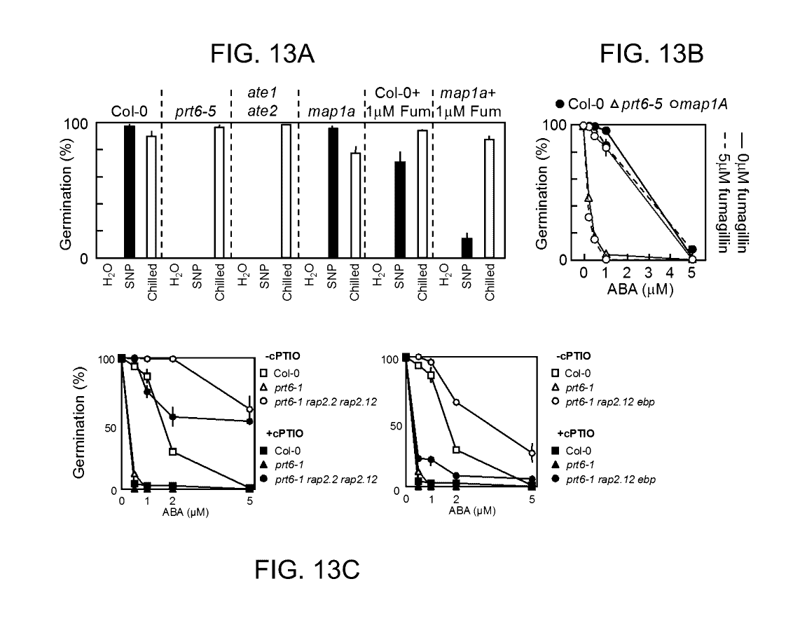

The data presented here, and in particular with reference to FIGS. 13A to 13C, demonstrates that by manipulating the N-end rule pathway the germination performance of seeds can be controlled. The data presented here shows that the MC-ERF transcription factors RAP2.2, RAP2.12 and EBP (RAP2.3) exert control on germination and seed dormancy by controlling ABA sensitivity.

### Example

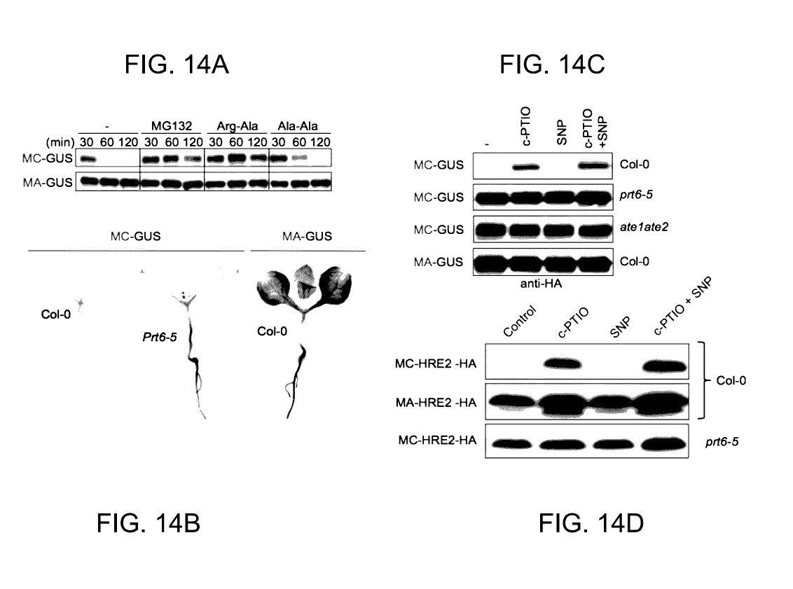

The data presented in FIG. 14 demonstrates that the accumulation of MC-initiating proteins can be manipulated by the levels of nitric oxide. The data shows that as well as oxygen, nitric oxide (NO) can control the stability of MC-initiating proteins. This is demonstrated using the artificial reporter MC-GUS and the endogenous substrate MC-ERF HRE2.

### REFERENCES

1 Bailey-Serres, J. & Voesenek, L. Flooding stress: Acclimations and genetic diversity. Annu. Rev. Plant Biol. 59, 313-339, (2008).
2 Kaelin, W. G. & Ratcliffe, P. J. Oxygen sensing by metazoans: The central role of the HIF hydroxylase pathway. Molecular Cell 30, 393-402, (2008).
3 Xu, K. et al. Sub1A is an ethylene-response-factor-like gene that confers submergence tolerance to rice. Nature 442, 705-708, (2006).
4. Fukao, T., Yeung, E. & Bailey-Serres, J. The Submergence Tolerance Regulator SUB1A Mediates Crosstalk between Submergence and Drought Tolerance in Rice. Plant Cell 23, 412-427, (2011).
5 Bachmair, A., Finley, D. & Varshaysky, A, In vivo half-life of a protein is a function of its amino-terminal residue. Science 234, 179-186, (1986).
6 Varshaysky, A. Regulated protein degradation. Trends in Biochem. Sci. 30, 283-286, (2005).
7 Kwon, Y. T. et al. An essential role of N-terminal arginylation in cardiovascular development. Science 297, 96-99, (2002).
8 Graciet, E., Mesiti, F. & Wellmer, F. Structure and evolutionary conservation of the plant N-end rule pathway. Plant J. 61, 741-751, (2010).
9 Graciet, E. & Wellmer, F. The plant N-end rule pathway: structure and functions. Trends Plant Sci. 15, 447-453, (2010).
10 Garzon, M. et al. PRT6/At5g02310 encodes an Arabidopsis ubiquitin ligase of the N-end rule pathway with arginine specificity and is not the CER3 locus. Febs Letters 581, 3189-3196, (2007).
11 Tasaki, T. & Kwon, Y. T. The mammalian N-end rule pathway: new insights into its components and physiological roles. Trends in Biochem. Sci. 32, 520-528, (2007).
12 Holman, T. J. et al. The N-end rule pathway promotes seed germination and establishment through removal of ABA sensitivity in Arabidopsis. PNAS (USA) 106, 4549-4554, (2009).
13 Yoshida, S., Ito, M., Callis, J Nishida, I. & Watanabe, A. A delayed leaf senescence mutant is defective: in arginyl-tRNA: protein arginyltransferase, a component of the N-end rule pathway in Arabidopsis. Plant J. 32, 129-137, (2002).
14 Graciet, E. et al. The N-end rule pathway controls multiple functions during. Arabidopsis shoot and leaf development. PNAS (USA) 106, 13618-13623, (2009).
15. Mustroph, A. et al. Profiling translatomes of discrete cell populations resolves altered cellular priorities during hypoxia in Arabidopsis. PNAS (USA) 106, 18843-18848, (2009).
16 Chung, H. J. & Feri, R. J. Arabidopsis alcohol dehydrogenase expression in both shoots and roots is conditioned by root growth environment. Plant Physiol. 121, 429-436, (1999).
17 Nakano, T., Suzuki, K., Fujimura, T. & Shinshi, H. Genome-wide analysis of the ERF gene family in Arabidopsis and rice. Plant Physiol. 140, 411-432, (2006).
18 Licausi, F. et al. HRE1 and HRE2, two hypoxia-inducible ethylene response factors, affect anaerobic responses in Arabidopsis thaliana. Plant 62, 302-315, (2010).
19 Hinz, M. et al. Arabidopsis RAP2.2: An Ethylene Response Transcription Factor That Is Important for Hypoxia Survival. Plant Physiol. 153, 757-772, (2010).
20 Papdi, C. et al. Functional identification of Arabidopsis stress regulatory genes using the controlled cDNA overexpression system. Plant Physiol. 147, 528-542, (2008).
21 Hattori, Y, et al. The ethylene response factors SNQRKEL1 and SNORKEL2 allow rice to adapt to deep water. Nature 460, 1026-U1116, (2009).
22 Hu, R. G. et al. The N-end rule pathway as a nitric oxide. sensor controlling the levels of multiple regulators. Nature 437, 981-986, (2005).
23 Lee, M. J. et al. RGS4 and RGS5 are in vivo substrates of the N-end rule pathway. PNAS (USA) 102, 15030-15035, (2005).
24 Takahashi, H. et al. A simple and high-sensitivity method for analysis of ubiquitination and polyubiquitination based on wheat cell-free protein synthesis. BMC Plant Biol. 9, 11, (2009).
25 Suzuki, T. & Varshaysky, A. Degradation signals in the lysine-asparagine sequence space. EMBO Journal 18, 6017-6026, (1999).
26 Leonard, S. E. & Carroll, K. S. Chemical ‘omics’ approaches for understanding protein cysteine oxidation in biology. Curr. Qpin. Chem. Biol. 15, 88-102, (2011).
27 Felle, H. H. pH regulation in anoxic plants. Ann. Bot. 96, 519-532, (2005).
28 Hu, R. G., Wang, H. Q., Xia, Z. X. & Varshaysky, A. The N-end rule pathway is a sensor of heme. PNAS (USA) 105, 76-81, (2008).
29 Côme, D. & Tissaoui, T. Induction dune dormance embryonnaire secondaire chez le pommier (Pinus malus L.) par des atmosphères très appauvries en oxygè\grave{n}e. Comptes Rendus de l'Acadèmie des Sciences, Paris 266, 477-479, (1968).
30 Branco-Price, C., Kaiser, K. A., Jang, C. J. H., Larive, C. K. & Bailey-Serres, J. Selective mRNA translation coordinates energetic and metabolic adjustments to cellular oxygen deprivation and reoxygenation in Arabidopsis thaliana. Plant J. 56, 743-755, (2008).
31 Mustroph, A. et al. Cross-Kingdom Comparison of Transcriptomic Adjustments to Low-Oxygen Stress Highlights Conserved and Plant-Specific Responses. Plant Physiol. 152, 1484-1500, (2010).
32 Lucas, M. et al. SHORT-ROOT Regulates Primary, Lateral, and Adventitious Root Development in Arabidopsis. Plant Physiol. 155, 384-398, (2011).
33 Dubin, M. J., Bowler, C. & Benvenuto, G. A modified Gateway cloning strategy for overexpressing tagged proteins in plants. Plant Methods 4, 11, (2008).
34 Swarup, R. et al. Root gravitropism requires lateral root cap and epidermal cells for transport and response to a mobile auxin signal. Nat. Cell Biol. 7, 1057-1065, (2005).
35 Martinez-Garcia, J. F., Monte, E. & Quail, P. H. A simple, rapid and quantitative method for preparing Arabidopsis protein extracts for immunoblot analysis. Plant J. 20, 251-257, (1999).
36 Williams, A. J., Werner-Fraczek, J., Chang, I. F. & Bailey-Serres, J. Regulated phosphorylation of 40S ribosomal protein S6 in root tips of maize. Plant Physiol. 132, 2086-2097, (2003).
37 Thompson, J. D., Higgins, D. G. & Gibson, T. J. Clustal-W—Improving the Sensitivity of Progressive Multiple Sequence Alignment Through Sequence Weighting, Position-Specific Gap Penalties and Weight Matrix Choice. Nucleic Acids Research 22, 4673-4680, (1994).
38 Horan, K. et al. Annotating genes of known and unknown function by large-scale coexpression analysis. Plant Physiol. 147, 41-57, (2008).

What is claimed is:

1. A method of improving plant growth characteristics comprising the steps of:
(i) disrupting the N-end rule pathway of targeted proteolysis in the plant; and
(ii) selecting for plants having improved growth characteristics.

2. The method of claim 1, wherein the N-end rule pathway of targeted proteolysis is disrupted in a manner selected from the group consisting of:
reducing or eliminating the ability of the N-end rule pathway of targeted proteolysis to degrade proteins initiating with the MC-motif at the N-terminus;
modifying MC-motif proteins so that they are no longer substrates for the N-end rule pathway of targeted proteolysis;
increasing the level of expression of the substrates of the N-end rule pathway of targeted proteolysis such that the level of un-degraded protein is elevated; and
altering the level of oxygen and/or nitric oxide to which the plant is exposed.

3. The method of claim 1, wherein the N-end rule pathway of targeted proteolysis is disrupted by modifying the MC-motif of one or more MC-motif proteins so that they are not substrates for the N-end rule pathway of targeted proteolysis.

4. The method of claim 3 wherein one or more modified MC-motif proteins are provided exogenously to the cell.

5. The method of claim 3, wherein the MC-motif protein is an ethylene response factor (ERF) Group VII transcription factor.

6. The method of claim 5, wherein the ERF Group VII transcription factor is selected from the group consisting of:
HRE1, a homolog, and a species ortholog thereof;
HRE2, a homolog, and a species ortholog thereof;
RAP2.12, a homolog, and a species ortholog thereof;
RAP2.2, a homolog, and a species ortholog thereof; and
RAP2.3(EBP), a homolog, and a species ortholog thereof.

7. The method of claim 1, wherein the plant is selected from the group consisting of: rice, wheat, tobacco, corn, and soy.

8. The method of claim 1, wherein the improved plant growth characteristic is selected from the group consisting of:
resistance to oxygen stress/hypoxia;
tolerance to drought conditions;
improved water use efficiency;
lower stomal density and/or index;
improved germination performance;
improved dormancy performance;
increased or decreased ABA sensitivity; and
improved resistance to abiotic stresses.

9. A plant obtained by the method of claim 1.

10. A genetically modified plant cell comprising improved plant growth characteristics and a disrupted N-end rule pathway of targeted proteolysis.

11. The plant cell of claim 10, wherein the N-end rule pathway of targeted proteolysis is disrupted in a manner selected from the group consisting of:
reducing or eliminating the ability of the N-end rule pathway of targeted proteolysis to degrade proteins initiating with the MC-motif at the N-terminus;
modifying MC-motif proteins so that they are no longer substrates for the N-end rule pathway of targeted proteolysis;
increasing the level of expression of the substrates of the N-end rule pathway of targeted proteolysis such that the level of un-degraded protein is elevated; and
altering the level of oxygen and/or nitric oxide to which the plant is exposed.

12. The plant cell of claim 10, wherein the N-end rule pathway of targeted proteolysis is disrupted by modifying the MC-motif of one or more MC-motif proteins so that they are not substrates for the N-end rule pathway of targeted proteolysis.

13. The plant cell of claim 12 wherein one or more modified MC-motif proteins are provided exogenously to the cell.

14. The plant cell of claim 12, wherein the MC-motif protein is an ethylene response factor (ERF) Group VII transcription factor.

15. The plant cell of claim 14, wherein the ERF Group VII transcription factor is selected from the group consisting of:
HRE1, a homolog, and a species ortholog thereof;
HRE2, a homolog, and a species ortholog thereof;
RAP2.12, a homolog, and a species ortholog thereof;
RAP2.2, a homolog, and a species ortholog thereof; and
RAP2.3(EBP), a homolog, and a species ortholog thereof.

16. The plant cell of claim 10, wherein the plant is selected from the group consisting of: rice, wheat, tobacco, corn, and soy.

17. The plant cell of claim 10, wherein the improved plant growth characteristic is selected from the group consisting of:
resistance to oxygen stress/hypoxia;
tolerance to drought conditions;
improved water use efficiency;
lower stomal density and/or index;
improved germination performance;
improved dormancy performance;
increased or decreased ABA sensitivity; and
improved resistance to abiotic stresses.

18. An expression cassette comprising a promoter operably linked to a polynucleotide encoding a stabilized form of an ethylene response factor (ERF) Group VII transcription factor.

19. The expression cassette of claim 18, wherein the ERF Group VII transcription factor is selected from the group consisting of:
HRE1, a homolog, and a species ortholog thereof;
HRE2, a homolog, and a species ortholog thereof;
RAP2.12, a homolog, and a species ortholog thereof;
RAP2.2, a homolog, and a species ortholog thereof; and
RAP2.3(EBP), a homolog, and a species ortholog thereof.
